(19) United States
(12) Patent Application Publication
(10) Pub. No.: US 2007/0112175 A1
(43) Pub. Date: May 17, 2007

(54) Determining drug resistance

(75) Inventor: Maria Kavallaris, Randwick (AU)

Correspondence Address: DAVIS WRIGHT TREMAINE LLP, 865 FIGUEROA STREET, SUITE 2400, LOS ANGELES, CA, 90017-2566, US

(73) Assignee: Children's Cancer Institute Australia for Medical Research, Randwick (AU)

(21) Appl. No.: 10/549,831

(22) PCT Filed: Mar. 18, 2004
(86) PCT No.: PCT/AU04/00331

§ 371 Date: Nov. 30, 2006

(30) Foreign Application Priority Data
Mar. 18, 2003 (AU) 2003901239

(51) Int. Cl. A61K 38/55; C07H 21/04; C12P 21/06; C07K 14/47

(52) U.S. Cl. 530/350; 435/69.1; 435/320.1; 435/325; 536/23.5; 514/12

(57) ABSTRACT

A method for inducing in a cell a resistance to an anti-microtubule agent comprising the step of providing in a cell, a nucleic acid molecule that encodes a mutant actin, or providing in a cell, a mutant actin.

This application is the National Phase of International Application PCT/AU2004/000331, filed Mar. 18, 2004, which designated the U.S. and that International Application was published under PCT Article 21(2) in English. This application also includes a claim of priority under 35 U.S.C. §119(a) and §365(b) to Australian patent application No. AU2003901239, filed Mar. 18, 2003.

### TECHNICAL FIELD OF THE INVENTION

The invention relates to mutations of the γ actin gene, to determining whether an individual is resistant to compounds for therapy of cancer by reference to the mutations, and to use of the mutations for inducing in a cell a resistance to such compounds.

### BACKGROUND OF THE INVENTION

Vinblastine is a compound used for the therapy of cancer. Desoxyepothilone B is a new experimental compound that has shown good efficacy in experimental animal models. These compounds are anti-microtubule agents and they provide therapy by inducing cancer cell death.

While the majority of patients treated with vinblastine ultimately become long term survivors, a significant proportion of patients develop resistance to cell death induced by these compounds and consequently relapse. Anti-microtubule agents such as vinblastine and desoxyepothilone, are potent inhibitors of cell division.

Although resistance to compounds for therapy of cancer may be mediated by molecules such as ABC gene superfamily members (P-glycoprotein, MRP-1 and 2, MDR-1), or β-tubulin mutations, there appears to be other mechanism(s) that control resistance. It is believed that a mechanism that is independent of P-glycoprotein activity is important for controlling resistance to cell death induced by vinblastine and desoxyepothilone B. The nature of this mechanism(s) is not understood.

A consequence of the paucity of information in relation to the mechanism(s) for resistance to anti-microtubule agents such as vinblastine and desoxyepothilone B is that a limitation applies to the extent to which resistance to these agents can be determined. Accordingly, to date it has been difficult to identify an individual who is resistant to vinblastine and/or desoxyepothilone therapy, or predisposed to resistance to such therapy.

Further it has been difficult to confer a resistance to anti-microtubule agents such as vinblastine or desoxyepothilone B upon a cell.

### SUMMARY OF THE INVENTION

The invention seeks to at least minimise the above identified limitations and in one aspect provides a method for determining whether a human cell is resistant to cell death induced by an anti-microtubule agent such as vinblastine and desoxyepothilone B. The method comprises determining whether the cell comprises a mutation of the γ actin gene or a mutated γ actin protein.

The invention further provides a method for determining whether a human cell is resistant to cell death induced by vinblastine. The method comprises determining whether the cell comprises a guanine (G) or cytosine (C), at position 559 of the γ actin cDNA, or G or thymine (T), at position 307 of the γ actin cDNA.

The nucleotides of the γ actin cDNA described herein are numbered according to the numbering of the sequence shown in SEQ ID NO: 1.

The invention further provides a method for determining whether a human cell is resistant to cell death induced by desoxyepothilone B. The method comprises determining whether the cell comprises a C or T, at position 293 of the γ actin cDNA, or C or T at position 485 of the γ actin cDNA.

As described herein, the inventors have characterised gene and protein expression in cell lines made resistant to vinblastine or desoxyepothilone B. More particularly, the inventors have isolated novel isoforms of γ actin and they have characterised the amino acid sequences of these isoforms and the nucleotide sequence of the cDNA encoding these isoforms. The inventors have found that particular mutations of the γ actin gene are associated with resistance to cell death induced by vinblastine or desoxyepothilone B and other anti-microtubule agents. This finding is unanticipated because vinblastine and desoxyepothilone B target β tubulin.

The inventors have found two mutations of the γ actin gene in cell lines made resistant to vinblastine as follows: (i) a mutation which controls a aspartate(D)¹⁸⁷ to histidine(H)¹⁸⁷ substitution in vinblastine resistant cells and (ii) a mutation which controls a valine(V)¹⁰³ to leucine(L)¹⁰³ substitution in vinblastine resistant cells. Further, the inventors have found two mutations in desoxyepothilone B resistant cell lines as follows: (i) a mutation which controls a proline(P)⁹⁸ to leucine(L)⁹⁸ substitution in desoxyepothilone B resistant cells and (ii) a mutation which controls a threonine(T)¹⁶² to methionine(M)¹⁶² substitution in desoxyepothilone B resistant cells.

The amino acids of γ actin described herein are numbered according to the numbering of the sequence shown in SEQ ID NO: 5.

As described herein, the mutations of the γ actin gene encode amino acid substitutions in regions of γ actin that are closely spatially related. More particularly, the substitutions are located in sub domain I and sub domain IV of γ actin. Importantly, it is recognised that these amino acid substitutions may indeed control or contribute to resistance to anti-microtubule agents such as vinblastine and desoxyepothilone B, because they are located either in close spatial relationship to the ATP binding cleft of sub domain IV, or in sub domain I. The inventors recognise that whether or not the mutations control or contribute to anti-microtubule agent resistance; as these mutations are observed to at least associate with the resistance to these compounds, they are useful for determining whether a cell is resistant to anti-microtubule agents such as vinblastine and desoxyepothilone B and accordingly, the identification of an individual who is resistant to therapy provided by these compounds.

Thus in another aspect, the invention provides a method for identifying an individual who is resistant to cell death induced by an anti-microtubule agent such as vinblastine and desoxyepothilone B or pre-disposed to resistance to cell death induced by such agents. The method comprises determining whether a cell of the individual comprises a mutation of the γ actin gene or a mutated γ actin protein.

In another aspect, the invention provides a method for identifying an individual who is resistant to cell death induced by vinblastine, or predisposed to resistance to cell death induced by vinblastine. The method comprises determining whether a cell derived from the individual comprises a G or C, at position 559 of the γ actin cDNA, or G or T, at position 307 of the γ actin cDNA.

In another aspect, the invention provides a method for identifying an individual who is resistant to cell death induced by desoxyepothilone B, or predisposed to resistance induced by desoxyepothilone B. The method comprises determining whether a cell derived from the individual comprises a C or T, at position 293 of the γ actin cDNA, or C or T, at position 485 of the γ actin cDNA.

Typically, the human cell the subject of the above described methods is a cancer cell, such as a leukemia. However, the inventors recognise that resistance to vinblastine or desoxyepothilone B may be detected in a normal human cell, more specifically a non cancerous or non neoplastic cell.

The inventors recognise that in particular embodiments, the above described methods could be performed, either by directly detecting the nucleotide at position 559, 307, 293 or 485 of the γ actin cDNA, or alternatively, by determining whether the cell or individual comprises at least one nucleotide in linkage disequilibrium with a nucleotide of the γ actin gene which corresponds to the nucleotide at position 559, 307, 293 or 485 of the γ actin cDNA. “Linkage disequilibrium” is known in the art as a phenomenon that occurs when the observed frequencies of haplotypes in a population does not agree with haplotype frequencies predicted by multiplying together the frequency of individual genetic markers in each haplotype. As discussed further herein, linkage disequilibrium occurs particularly when genes are located in close physical proximity, and/or when genomic DNA between genes is otherwise not susceptible to recombination.

As described herein, the inventor has found that the mutation of the γ actin gene, and the γ actin protein expressed therefrom, confer resistance to anti-microtubule agents upon a cell. Specifically, by introducing a nucleic acid molecule comprising the γ actin mutation into a cell, the sensitivity of the cell to agents such as vinblastine, desoxyepothilone B and paclitaxel can be minimised, decreased or otherwise adjusted and indeed a cell can be produced that has a level of resistance to these compounds. This finding has useful application particularly in the circumstance where there is a need to minimise or avoid the side effects that are observed in cancer therapy with these compounds.

Thus in another aspect, the invention provides a method for inducing, conferring or otherwise imparting to a cell, a resistance to an anti-microtubule agent. The method comprises the step of providing in a cell, a nucleic acid molecule that encodes a mutant γ actin, or providing in a cell, a mutant γ actin.

An anti-microtubule agent is a compound that binds to β tubulin. Typically anti-microtubule agents, on binding to β tubulin, disrupt microtubule dynamics. Such disruption may lead to mitotic arrest and/or cell death. Anti-microtubule agents are used extensively in the treatment of many types of cancer. In some circumstances, the clinical usefulness of anti-microtubule agents is limited by the development of resistance to these agents. Examples of anti-microtubule agents include vinblastine, desoxyepothilone B, vincristine and paclitaxel.

In another aspect, the invention provides a method for producing a cell that is resistant to an anti-microtubule agent comprising the step of providing in a cell, a nucleic acid molecule that encodes a mutant γ actin, or providing in a cell, a mutant γ actin.

In another aspect, the invention provides a method for treating an individual for cancer with an anti-microtubule agent comprising the step of providing in a non cancerous cell of the individual, a nucleic acid molecule that encodes a mutant γ actin, or providing in a non cancerous cell, a mutant γ actin, to induce in the non cancerous cell, a resistance to an anti-microtubule agent.

In another aspect, the invention provides a method for determining whether a compound is capable of treating a cell having a drug resistance phenotype comprising the steps of:

providing in a cell a nucleic acid molecule that encodes a mutant γ actin, or providing in a cell, a mutant γ actin, to induce in the cell, a resistance to an anti-microtubule agent; and

contacting the cell with the compound to determine whether the compound is capable of treating the cell. A cell having a drug resistance phenotype is typically one that has become resistant to treatment with a cancer chemotherapeutic. The cell phenotype may be characterised by the expression of molecules such as p-glycoprotein or mutated β tubulin, or mutated γ actin.

In another aspect, the invention provides a peptide for inducing in a cell a resistance to an anti-microtubule agent comprising:

(a) a sequence shown in any one of SEQ ID NOS: 5 to 8; or

(b) a sequence that has at least 80% identity to the sequence shown in SEQ ID NO: 5 and wherein residue number 187 is histidine; or

(c) a sequence that has at least 80% identity to the sequence shown in SEQ ID NO: 6 and wherein residue number 103 is leucine; or

(d) a sequence that has at least 80% identity to the sequence shown in SEQ ID NO: 7 and wherein residue number 98 is leucine; or

(e) a sequence that has at least 80% identity to the sequence shown in SEQ ID NO: 8 and wherein residue number 162 is methionine.

(f) a fragment of a peptide according to any one of (a) to (e).

The peptide is useful for determining whether a human cell or individual is resistant to cell death induced by vinblastine or desoxyepothilone B. It is also useful for inducing, conferring or otherwise imparting to a cell a resistance to an anti-microtubule agent such as vinblastine and desoxyepothilone B.

The invention also provides γ actin that comprises one of the following sequences: (i) DLTHYLMK (SEQ ID NO: 9); or (ii) VAPEEHPVLLTEAPLNPK (SEQ ID NO: 10); or histidine at amino acid position 187 or leucine at amino acid position 103 or leucine at amino acid position no 98 or methionine at amino acid position no. 162.

The invention also provides a fragment of a γ actin described above useful for immunising a host to produce an antibody for determining whether a human cell or individual is resistant to cell death induced by vinblastine or desoxyepothilone B.

In another aspect, the invention provides a nucleic acid molecule for inducing in a cell a resistance to an anti-microtubule agent comprising:

(a) a sequence shown in any one of SEQ ID NOS: 1 to 4; or

(b) a sequence that has at least 80% identity to the sequence shown in SEQ ID NO: 1 and wherein nucleotide number 559 is cytosine; or

(c) a sequence that has at least 80% identity to the sequence shown in SEQ ID NO: 2 and wherein nucleotide number 307 is thymine; or

(d) a sequence that has at least 80% identity to the sequence shown in SEQ ID NO: 3 and wherein nucleotide number 293 is thymine; or

(e) a sequence that has at least 80% identity to the sequence shown in SEQ ID NO: 4 and wherein nucleotide number 485 is thymine; or

(f) a fragment of a molecule according to any one of (a) to (e).

The nucleic acid molecule is useful for determining whether a human cell or individual is resistant to cell death induced by vinblastine or desoxyepothilone B. It is also useful for inducing, conferring or otherwise imparting to a cell a resistance to an anti-microtubule agent such as vinblastine and desoxyepothilone B.

In another aspect, the invention provides a vector comprising a nucleic acid molecule according to the invention.

In another aspect, the invention provides a cell comprising a peptide, nucleic acid molecule or vector according to the invention.

In another aspect, the invention provides an antibody for binding to a peptide or a γ actin of the invention. The antibody is useful for determining whether a human cell or individual is resistant to cell death induced by vinblastine or desoxyepothilone B.

### DETAILED DESCRIPTION OF THE INVENTION

As described further herein, the inventors have found a number of mutations at various position of the γ actin cDNA which are associated with resistance to cell death induced by vinblastine, or resistance to cell death induced by desoxyepothilone B. As these mutations are passed on subsequent to mitotic cell division, it follows that they are mutations of genomic DNA.

More particularly, the inventors have identified that in cells resistant to vinblastine, the nucleotide at position 559 of the γ actin cDNA is C and encodes H at 187, while in cells sensitive to vinblastine, the nucleotide at position 559 of the cDNA is G and encodes D. A further mutation is that, in cells resistant to vinblastine, the nucleotide at position 307 of the γ actin cDNA is T and encodes L at position 103, whereas in cells sensitive to vinblastine, the nucleotide at position 307 of the cDNA is G and encodes V. As the cells sensitive to vinblastine have G at position 559 or 307 of the γ actin cDNA, the presence of G at position 559 or 307 is considered to be and is described herein as a wild type (wt) genotype. The presence of C at position 559 or a T at position 307 is considered to be, and is described herein as mutant (m1) and (m2) genotypes respectively.

Further, the inventors have identified that in cells resistant to desoxyepothilone B, the nucleotide at position 293 of the γ actin cDNA is T and encodes L at 98, while in cells sensitive to desoxyepothilone B, the nucleotide at position 293 of the cDNA is C and encodes P. A further mutation is that, in cells resistant to desoxyepothilone B, the nucleotide at position 485 of the γ actin cDNA is T and encodes M at position 162, whereas in cells sensitive, the nucleotide at position 485 of the cDNA is C and encodes T. As the cells sensitive have C at position 293 or 485 of the γ actin cDNA, the presence of C at position 293 or 485 is considered to be and is described herein as a wild type (wt) genotype. The presence of T at position 293 or 485 is considered to be, and is described herein as mutant genotypes, m3 and m4 respectively.

As described herein, it is recognised that sensitivity or resistance to vinblastine or desoxyepothilone B could be detected by directly detecting the above identified mutations. Alternatively, this could be detected by detecting one or more alleles in linkage disequilibrium with a corresponding above identified mutation.

There now follows a description of methods, nucleic acid molecules, peptides, antibodies, composition and kits for determining whether a human cell or individual is resistant to cell death induced by vinblastine or desoxyepothilone B and for inducing, conferring or otherwise imparting to a cell a resistance to an anti-microtubule agent such as vinblastine and desoxyepothilone B.

### A. Methods

### a. Methods for Identifying γ Actin

The identity of the γ actin can be determined by use of any technique capable of distinguishing between the wt gene product and the m1, m2, m3 or m4 gene products.

In one embodiment, the method comprises contacting the γ actin isoform with an antibody for selectively binding to a region of the isoform comprising amino acid residue position 103 or 187 in conditions for permitting the antibody to bind to the region. The binding of the antibody to an isoform comprising H at position 187 or L residue at 103 identifies that the isoform is a m1 or m2 gene product respectively. Accordingly, a cell to which the antibody is bound is resistant to vinblastine therapy.

In another embodiment, the method comprises contacting the γ actin isoform with an antibody for selectively binding to a region of the isoform comprising amino acid residue position 162 or 98 in conditions for permitting the antibody to bind to the region. The binding of the antibody to an isoform comprising L at position 98 or M residue at 162 identifies that the isoform is a m3 or m4 gene product respectively. Accordingly, a cell to which the antibody is bound is resistant to desoxyepothilone B therapy.

Suitable assay formats for identification of the γ actin isoform include ELISA, radio-immunoassay, Western blots, immunofluorescence staining, immunoprecipitation, mass spectrometry, protein sequencing, gel electrophoresis and immunohistochemistry.

### b. Methods for Identifying a Mutation

1. Restriction Endonuclease Techniques

In one embodiment, the method comprises cleaving a nucleic acid of the cell with a restriction endonuclease for distinguishing G from C at position 559, or cleaving a nucleic acid of the cell with a restriction endonuclease for distinguishing G from T at position 307, for the purpose of determining resistance to vinblastine. The endonuclease may be one capable of cleaving at position 559 or 307 of γ actin, or it may be one for cleaving at a nucleotide in linkage disequilibrium with the nucleotide corresponding to the nucleotide at position 559 or 307 of the γ actin cDNA. The resultant cleaved or uncleaved products may then be detected as described further herein.

In another embodiment, and for the purpose of determining resistance to desoxyepothilone B, the method comprises cleaving a nucleic acid of the cell with a restriction endonuclease for distinguishing C from T at position 293, or cleaving a nucleic acid of the cell with a restriction endonuclease for distinguishing C from T at position 485. The endonuclease may be one capable of cleaving at position 293 or 485 of γ actin, or it may be one for cleaving at a nucleotide in linkage disequilibrium with the nucleotide corresponding to the nucleotide at position 293 or 485 of the γ actin cDNA. The resultant cleaved or uncleaved products may then be detected as described further herein.

The method is typically performed under optimal conditions for cleavage. These conditions are dictated by the type and specificity of the restriction endonuclease. In general, the conditions that are optimal for cleavage are those described in the product material with the manufacturer's instructions. Conditions for cleavage may modified by adjusting, for example, pH and salt concentrations of the reaction mixture.

The presence of the cleaved or uncleaved products may be detected by electrophoresis through agarose or polyacrylamide gels. This is a standard method used to separate and identify nucleic acid molecules. The technique is rapid and simple to perform. Nucleic acid molecules applied to the agarose or polyacrylamide gel are separated electrophoretically based upon their molecular weight.

The position of the cleaved or uncleaved product, and therefore, its identification, can be determined by staining of the product with a fluorescent intercalating dye such as ethidium bromide and then examination under ultraviolet light. Thus the molecular weights of the cleaved or uncleaved products can be determined by comparison of the migration of the products to commercially available molecular weight standards.

Alternatively, the cleaved and uncleaved products may be arranged onto a solid support, for example, a nitrocellulose membrane, especially by capillary blotting. The products are then identified by hybridising a nucleic acid molecule (a “probe”) to a portion of the cleaved or uncleaved product that comprises sequence (the “target sequence”) that is complementary to the probe, and that is arranged on the support. These methods are described further herein. Methods for the design and production of a probe are also described further herein.

The mutation C to T at position 293 generates a unique restriction site with BsiL1 resulting in 5 fragments in the mutant allele in contrast to four fragments from the wild type allele when digesting a PCR product encompassing nucleotides 1 to 654. The mutation C to T at position 485 generates a unique restriction site with BscB1 resulting in 8 fragments in the mutant allele in contrast to 7 fragments from the wild type allele when digesting a PCR product encompassing nucleotides 1 to 654.

Other endonucleases may be used in the method particularly where the DNA of the individual is not in fact genomic DNA, but is an amplification product obtained by amplification of the individual's DNA, for example by the polymerase chain reaction (PCR) as described further herein, and a particular restriction site corresponding with the nucleotide at position 559, 307, 293 or 485 is incorporated into the amplified product by a PCR primer.

In accordance with the preceding paragraph, it will be understood that the actual material to be determined in the technique may be a sample of genomic DNA from the individual, or a sample of another nucleic acid molecule, i.e. DNA, cDNA or RNA or a mixture thereof, derived, for example, by amplification in a PCR, from a sample of the individual's DNA.

It follows therefore, that the restriction endonuclease technique may be preceded by amplification of the individual's DNA. Amplification methods, including PCR, are described further herein.

2. Nucleotide Sequence Techniques

In one embodiment, the method comprises sequencing a portion of the nucleic acid of the cell for the purpose of determining whether the cell is resistant to vinblastine therapy. The portion may be one which comprises the nucleotide at position 559 or 307 and/or it may comprise a nucleotide in linkage disequilibrium with a nucleotide of the γ actin gene which corresponds with the nucleotide at position 559 or 307 of the γ actin cDNA.

In another embodiment, the method comprises sequencing a portion of the nucleic acid of the cell for the purpose of determining whether the cell is resistant to desoxyepothilone B therapy. The portion may be one which comprises the nucleotide at position 293 or 485 and/or it may comprise a nucleotide in linkage disequilibrium with a nucleotide of the γ actin gene which corresponds with the nucleotide at position 293 or 485 of the γ actin cDNA.

This technique combines (a) physio-chemical techniques, based on the denaturation and hybridisation of a nucleic acid molecule (a “primer”) to a sequence arranged in the individuals nucleic acid (the “target sequence”) that comprises sequence complementary to the primer sequence and (b) enzymatic reactions with endonucleases, ligases and/or polymerases. An exemplary method is the Sanger sequencing method.

The factors for consideration in determining appropriate primers for the methods of the invention are described further herein. Examples of suitable primers are those that span the entire length of the gene in both forward and reverse directions and are specific to the γ-actin sequence.

The primers may be labelled, and methods for labelling primers and examples of suitable labels are described further herein.

It will be understood that either or both of coding or non-coding strands may be sequenced. Accordingly, primers may be designed for hybridising to target sequences arranged in either the coding or non-coding strands.

The nucleotide sequence of a portion of the individual's nucleic acid is identified by separating fragments generated in a sequencing reaction on the basis of molecular weight. Polyacrylamide gel electrophoresis is an example of a suitable method.

The sequencing method can be applied when a sequence in the genomic DNA is known, such as where the primer hybridizes to a known γ actin target sequence and initiates primer extension into a known region of DNA for sequencing purposes. Alternatively, the method can be applied where previous sequencing has determined a region of nucleotide sequence and the primer is designed to extend from the determined region into a region of unknown sequence. This latter technique is particularly useful where the nucleotide to be detected is one in linkage disequilibrium with one of the above described mutations.

It will be understood that the actual material to be sequenced may be a sample of genomic DNA of the individual, or a sample of nucleic acid molecule, i.e. DNA, cDNA, RNA or a mixture thereof, derived, for example, amplified by PCR, from a sample of the individual's nucleic acid.

It follows therefore that the nucleotide sequencing technique may be preceded by amplification of the individual's nucleic acid. Amplification methods, including PCR, are described further herein.

3. Hybridisation Techniques

In another embodiment, a nucleic acid molecule capable of distinguishing G from C at position 559, or capable of distinguishing G from T at position 307, is hybridised with nucleic acid of the individual, to determine whether the cell is resistant to vinblastine therapy. The nucleic acid molecule may hybridise to a portion of the nucleic acid comprising the nucleotide at position 559 or 307 and/or it may hybridise to a portion comprising a nucleotide in linkage disequilibrium with a nucleotide of the γ actin gene corresponding to the nucleotide at position 559 or 307 of the γ actin cDNA.

In another embodiment, a nucleic acid molecule capable of distinguishing C from T at position 293, or capable of distinguishing C from T at position 485, is hybridised with nucleic acid of the individual, to determine whether the cell is resistant to desoxyepothilone B therapy. The nucleic acid molecule may hybridise to a portion of the nucleic acid comprising the nucleotide at position 293 or 485 and/or it may hybridise to a portion comprising a nucleotide in linkage disequilibrium with a nucleotide of the γ actin gene corresponding to the nucleotide at position 293 or 485 of the γ actin cDNA.

This technique is based on the denaturation and hybridisation of the nucleic acid molecule (the “probe”) and a sequence arranged on the individual's nucleic acid that comprises sequence complementary to the sequence of the probe (the “target sequence”), resulting in the probe hybridising with the target sequence to form a duplex.

Methods for the design and production of probes suitable for detecting the mutations are described further herein.

The conditions required for hybridisation of the probe to a target sequence are according to standard techniques. Briefly for hybridisation to occur between the probe and target sequence, appropriate hybridisation conditions must be provided. This requires a consideration of the concentration of probe and target sequence, the length of the probe and degree of complementarity between probe and target sequence, particularly, G/C content, the time allowed for hybridisation, temperature, and pH and salt concentration of the buffer.

Oligonucleotide probes ranging in length from 14 to 40 base pairs that specifically span each of the regions of the sequence of γ-actin at position 293, 307, 485, and 559. Probes will be labelled with a radioactive nucleotide or biotin-label for use in DNA or RNA hybridisation experiments. The probe sequences spanning the mutated regions that could be used are shown in the Table below.

| | | Hybridisation |
|---|---|---|
| Mutation | Probe Sequence | Temperature |
| M1 (D187H) | GGGTGTTCAAGGTCTCA | 55° C. |
| M2 (V103L) | GTCAGCAGCAATGGGTGCTC | 55° C. |
| M3 (P98L) | TGCTCCTCCAGGGCCAC | 65° C. |
| M4 (T162M) | GATGGGCACCATGTGGGT | 65° C. |

Similar probes containing wildtype sequence are also utilised. Radiolabelled probes are prepared as follows: 10 picomole of oligonucleotide probes, 10×polynucleotide buffer, ³²P-γ-dATP, and polynucleotide kinase are incubated in a final volume of 15 μl at 37° C. for 30 min followed by heat inactivation at 65° C. for 2 min. Membranes are hybridised with 2×10⁷ cpm/ml ³²P-labelled probe at 55-65° C. overnight followed by washing in 1×SSC buffer.

Hybridization can be carried out in a homogeneous or heterogeneous format. The homogeneous hybridization reaction occurs entirely in solution, in which both the probe and the target sequence are present in soluble forms in solution. A heterogeneous reaction involves the use of a matrix that is insoluble in the reaction medium to which either the probe or target sequence is bound.

Where the target sequence is in a double-stranded (ds) form, it is usually denatured as by heating or alkali treatment, prior to conducting the hybridization reaction. The denaturation of the ds target sequence can be carried out prior to admixture with the probe, or can be carried out after the admixture with the probe.

Effective amounts of probe for use in the hybridisation reaction are typically expressed in terms of molar ratios between the probe to be hybridized and the target sequence. Preferred ratios are hybridization reaction mixtures containing equimolar amounts of the target sequence and the probe. As is well known, deviations from equal molarity will produce duplexes, although at lower efficiency. Thus, although ratios where either probe or target sequence can be in as much as 100-fold molar excess relative to each other, excesses of less than 50-fold, preferably less than 10-fold, and more preferably less the 2-fold are desirable in practicing the invention.

The probe may include a label or indicating group that will render the probe and thus a duplex comprising the probe hybridised to the target sequence, detectable. The label may include radioactive elements, chemically modified nucleotide bases and the like.

Radioactive elements linked to a probe provide a useful means to facilitate the detection of target sequence and probe that have hybridised to form a duplex. A typical radioactive element is one that produces beta ray emissions. Elements that emit beta rays, such as ³H, ¹²C, ³²P and ³⁵S represent a class of suitable labels. A probe labelled with such a radioactive element is typically labelled by enzymic incorporation of the element, for example using DNA kinase.

Alternatives to radioactive labelled probes are probes that are chemically modified to contain metal complexing agents, biotin-containing groups, fluorescent compounds and the like.

One useful metal complexing agent is a lanthanide chelate formed by a lanthanide and an aromatic beta-diketone, the lanthanide being bound to the probe via a chelate forming compound such as an EDTA analogue so that a fluorescent lanthanide complex is formed.

Biotin or acridine ester-labelled probes have been described. Useful fluorescent marker compounds include fluorescein, rhodamine, Texas Red, NBD and the like.

A labelled probe present in a duplex comprising the target sequence renders the duplex itself labelled and therefore distinguishable over other nucleic acids presence in the sample to be assayed. Detecting the presence of the label in the duplex and thereby the presence of the duplex, typically involves separating the duplex from any labelled probe that is not hybridised to form a duplex. Preferred probes for use in forming a duplex are those comprising nucleotide sequences that span the regions of the mutations i.e. C at 559, T at 307, T at 293, and T at 485. Ideally these will span a region ranging from 14 to 40 bases.

Techniques for the separation of the probe, such as a non hybridised labelled probe, from a duplex, which are suitable, are those that separate single stranded and double stranded nucleic acid molecules on the basis of their chemical properties. An example is the use of a heterogenous hybridisation format in which the non hybridised probed is separated, typically by washing, from the duplex that is bound to an insoluble matrix.

The probe may also be linked, typically at or near the 5′-terminus to a solid support. Useful supports include cross-linked dextran, agarose, polystyrene or latex beads about 1 micron to about 5 mm in diameter, polyvinyl chloride, polystyrene, cross-linked polyacrylamide, nitrocellulose or nylon based webs such a sheets, strips etc.

Further examples are described below:

(1) Detection of Membrane Immobilized Target Sequences

In the Southern blot technique, specific regions of DNA are detected by immobilizing the target sequences on a membrane. The specific regions of DNA may be prepared by PCR amplification, by PCR amplification followed by digestion with restriction endonucleases, or by digestion with a restriction endonuclease without PCR amplification.

Accordingly, in one approach, DNA is first isolated. Specific regions of the DNA are then PCR amplified to generate target sequences that are then analyzed intact. Alternatively, the amplified target sequences are then subjected to restriction digestion. In another alternative, the DNA is cleaved by restriction endonucleases to form DNA fragments of discrete molecular weights.

The above-generated target sequences are then separated according to size in an agarose gel and transferred (blotted) onto a nitrocellulose or nylon membrane support. Conventional electrophoresis separates fragments ranging from 100 to 30,000 base pairs while pulsed field gel electrophoresis resolves fragments up to 20 million base pairs in length. The location on the membrane of a particular target sequence is then determined by direct visualization of stained DNA.

Target sequences may then be directly immobilized onto a solid-matrix (nitrocellulose membrane) using a dot-blot (slot-blot) apparatus, and analyzed by probe-hybridization.

Immobilized target sequences may be analyzed by probing with the probe. A probe is typically a synthetic DNA oligomer of approximately 20 nucleotides, preferably 17 nucleotides in length. A probe is long enough to represent unique sequences in the genome, but sufficiently short so that an internal mismatch of sequences of the probe and target sequence destabilises hybridization. Thus, any sequences differing at single nucleotides may be distinguished by the different hybridisation behaviors of probe and target sequences under carefully controlled hybridization conditions.

(2) Detection of Target Sequences in Solution

Several rapid techniques that do not require nucleic acid purification or immobilization are useful in the methods of the invention. For example, duplexes comprising the probe hybridised to the target sequence may be selectively isolated on a solid matrix, such as hydroxylapatite, which preferentially binds double-stranded nucleic acids. Alternatively, probe may be immobilized on a solid support and used to capture target sequences from solution. Detection of the target sequences can be accomplished with the aid of a second, labeled probe that is either displaced from the support by the target sequence in a competition type assay or joined to the support via the bridging action of the target sequence in a sandwich-type format.

Another suitable method is the oligonucleotide ligation assay (OLA) in which the enzyme DNA ligase is used to covalently join two synthetic oligonucleotide sequences selected so that they can base pair with a target sequence in exact head-to-tail juxtaposition. Ligation of the two oligomers is prevented by the presence of mismatched nucleotides at the junction region. This procedure allows for the distinction between known sequence variants in samples of cells without the need for DNA purification. The joint of the two oligonucleotides may be monitored by immobilizing one of the two oligonucleotides and observing whether the second, labeled oligonucleotide is also captured.

(3) Scanning Techniques for Detection of Base Substitutions

Three techniques permit the analysis of probe/target duplexes several hundred base pairs in length for unknown single-nucleotide substitutions or other sequence differences. In the ribonuclease (RNase) A technique, the enzyme cleaves a labeled RNA probe at position where it is mismatched to a target RNA or DNA sequence. The fragments may be separated according to size and the approximate position of the mutation identified.

In the denaturing gradient gel technique, a probe-target DNA duplex is analyzed by electrophoresis in a denaturing gradient of increasing strength. Denaturation is accompanied by a decrease in migration rate. A duplex with a mismatched base pair denatures more rapidly than a perfectly matched duplex.

A third method relies on chemical cleavage of mismatched base pairs. A mismatch between T and C, G, or T, as well as mismatches between C and T, A, or C, can be detected in heteroduplexes. Reaction with osmium tetroxide (T and C mismatches) or hydroxylamine (C mismatches) followed by treatment with piperidine cleaves the probe at the appropriate mismatch.

### d. Polymerase Chain Reaction Techniques

Polymorphic discrimination can be performed using either PCR based single nucleotide polymorphism analysis or by designing specific probes to be used in fluorescent PCR technology that allows for allellic discrimination. In both situations, primers/probes would amplify either the mutant or wild type γ-actin allele.

### B. Nucleic Acid Molecules

As described above, in another aspect, the invention provides a nucleic acid molecule for inducing in a cell a resistance to an anti-microtubule agent comprising:

(a) a sequence shown in any one of SEQ ID NOS: 1 to 4; or

(b) a sequence that has at least 80% identity, more preferably 85% identity, more preferably 90% identity, still more preferably 95% identity, yet more preferably 99% identity to the sequence shown in SEQ ID NO: 1 and wherein nucleotide number 559 is cytosine; or

(c) a sequence that has at least 80% identity more preferably 85% identity, more preferably 90% identity, still more preferably 95% identity, yet more preferably 99% identity to the sequence shown in SEQ ID NO: 2 and wherein nucleotide number 307 is thymine; or

(d) a sequence that has at least 80% identity more preferably 85% identity, more preferably 90% identity, still more preferably 95% identity, yet more preferably 99% identity to the sequence shown in SEQ ID NO: 3 and wherein nucleotide number 293 is thymine; or

(e) a sequence that has at least 80% identity more preferably 85% identity, more preferably 90% identity, still more preferably 95% identity, yet more preferably 99% identity to the sequence shown in SEQ ID NO: 4 and wherein nucleotide number 485 is thymine; or

(f) a fragment of a molecule according to any one of (a) to (e).

Nucleotide sequence identity can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

In one embodiment, the nucleic acid molecule comprises cytosine at nucleotide position 559 and is capable of hybridising to a nucleic acid molecule that is complementary to the sequence shown in SEQ ID NO:1 in high stringency conditions.

In one embodiment, the nucleic acid molecule comprises thymine at nucleotide position 307 and is capable of hybridising to a nucleic acid molecule that is complementary to the sequence shown in SEQ ID NO:2 in high stringency conditions.

In one embodiment, the nucleic acid molecule comprises thymine at nucleotide position 293 and is capable of hybridising to a nucleic acid molecule that is complementary to the sequence shown in SEQ ID NO:3 in high stringency conditions.

In one embodiment, the nucleic acid molecule comprises thymine at nucleotide position 485 and is capable of hybridising to a nucleic acid molecule that is complementary to the sequence shown in SEQ ID NO:4 in high stringency conditions.

By way of example, and not limitation, a procedure using conditions of high stringency is as follows: Prehybridization of filters containing DNA is carried out for 2 hours to overnight at 65° C. in buffer composed of 6×SSC, 5×Denhardt's solution, and 100 μg/ml denatured salmon sperm DNA. Filters are hybridized for 12 to 48 hrs at 65° C. in prehybridization mixture containing 100 μg/ml denatured salmon sperm DNA and 5−20×10⁶ cpm of ³² P-labeled probe. Washing of filters is done at 37° C. for 1 hr in a solution containing 2×SSC, 0.1% SDS. This is followed by a wash in 0.1×SSC, 0.1% SDS at 50° C. for 45 min. before autoradiography. Other procedures using conditions of high stringency would include either a hybridization step carried out in 5×SSC, 5×Denhardt's solution, 50% formamide at 42° C. for 12 to 48 hours or a washing step carried out in 0.2×SSPE, 0.2% SDS at 65° C. for 30 to 60 minutes.

Reagents mentioned in the foregoing procedures for carrying out high stringency hybridization are well known in the art. Details of the composition of these reagents can be found in, e.g., Sambrook et al., 1989, Molecular Cloning: A Laboratory Manual; Cold Spring Harbor Laboratory, Cold Spring Harbor, N.Y. in addition to the foregoing, other conditions of high stringency which may be used are well known in the art.

The nucleic acid molecule is useful for determining whether a human cell or individual is resistant to cell death induced by vinblastine or desoxyepothilone B. It is also useful for inducing, conferring or otherwise imparting to a cell a resistance to an anti-microtubule agent such as vinblastine and desoxyepothilone B.

The nucleic acid molecule may be a fragment of the molecule shown in any one of SEQ ID NOS: 1 to 4 provided that the fragment is capable of determining whether an individual or human cell is resistant to cell death induced by vinblastine or desoxyepothilone B. Such a fragment is one that includes a sequence that is characteristic of the M1, M2, M3, or M4 genotypes described herein. Suitable fragments are those of about 50 nucleotides in length.

The nucleic acid molecules may be produced by standard techniques including solid phase synthesis or recombinant techniques.

### C. Peptides

As described above, in one aspect, the invention provides a peptide for inducing in a cell a resistance to an anti-microtubule agent comprising:

(a) a sequence shown in any one of SEQ ID NOS: 5 to 10; or

(b) a sequence that has at least 80% identity more preferably 85% identity, more preferably 90% identity, still more preferably 95% identity, yet more preferably 99% identity to the sequence shown in SEQ ID NO: 5 and wherein residue number 187 is histidine; or

(c) a sequence that has at least 80% identity more preferably 85% identity, more preferably 90% identity, still more preferably 95% identity, yet more preferably 99% identity to the sequence shown in SEQ ID NO: 6 and wherein residue number 103 is leucine; or

(d) a sequence that has at least 80% identity more preferably 85% identity, more preferably 90% identity, still more preferably 95% identity, yet more preferably 99% identity to the sequence shown in SEQ ID NO: 7 and wherein residue number 98 is leucine; or

(e) a sequence that has at least 80% identity more preferably 85% identity, more preferably 90% identity, still more preferably 95% identity, yet more preferably 99% identity to the sequence shown in SEQ ID NO: 8 and wherein residue number 162 is methionine;

(f) a fragment of a peptide according to any one of (a) to (e).

The invention also provides γ actin that comprises one of the following sequences: (i) DLTHYLMK (SEQ ID NO: 9); or (ii) VAPEEHPVLLTEAPLNPK (SEQ ID NO: 10); or histidine at amino acid position 187 or leucine at amino acid position 103 or leucine at amino acid position no 98 or methionine at amino acid position no. 162.

The invention also provides a fragment of a γ actin described above useful for immunising a host to produce an antibody for determining whether a cell is resistant to cell death induced by vinblastine or desoxyepothilone B. Such fragments comprise histidine at amino acid position 187 or leucine at amino acid position 103 or leucine at amino acid position no 98 or methionine at amino acid position no. 162. Examples of suitable fragments are (i) DLTHYLMK (SEQ ID NO: 9) and (ii) VAPEEHPVLLTEAPLNPK (SEQ ID NO: 10).

The peptide and γ actin of the invention are useful for determining whether a human cell or individual is resistant to cell death induced by vinblastine or desoxyepothilone B. It is also useful for inducing, conferring or otherwise imparting to a cell a resistance to an anti-microtubule agent such as vinblastine and desoxyepothilone B.

The peptides, γ actin and fragments thereof, are particularly useful for the production of antibodies for determining whether a human cell or individual is resistant to cell death induced by vinblastine or desoxyepothilone B.

The peptide may consist of a portion of the sequence shown in any one of SEQ ID NOS: 5 to 10.

The peptides, γ actin or fragments thereof, may be produced by standard techniques including Merrifield synthesis or recombinant techniques.

### D. Antibodies

As described above, in one aspect, the invention provides an antibody for binding to a peptide, γ actin or fragments thereof the invention. The antibody is useful for determining whether a human cell or individual is resistant to cell death induced by vinblastine or desoxyepothilone B.

The antibody may be a monoclonal antibody or a polyclonal antibody, for example, a rabbit, goat, horse or donkey derived anti sera. Alternatively, the antibody may be derived by recombinant DNA technology. The antibody may be a chimeric antibody, for example, comprising both human and murine domains, or it may be a fAb, dAb, scfv or CDR.

Anti-sera to the peptides and γ actin of the invention can be raised by immunisation with the peptides, γ actin or fragments thereof, described herein according to standard techniques.

### E. Composition

The present invention also provides composition of nucleic acid molecules described above for determining whether an individual or human cell is resistant to vinblastine or desoxyepothilone B, or for inducing, conferring or otherwise imparting to a cell a resistance to an anti-microtubule agent such as vinblastine and desoxyepothilone B.

The composition may comprise at least one nucleic acid molecule for detecting any one of the mutations described above. The composition may be obtained by conventional nucleic acid procedures, including synthesis, isolation, purification, PCR amplification and the like.

Alternatively, the composition may comprise at least one peptide, γ actin or fragments thereof described above and a carrier, diluent or excipient.

Alternatively, the composition may comprise at least one antibody for selectively binding to a peptide, γ actin or fragments thereof described above and a carrier, diluent or excipient

### F. Kits

The present invention also provides a system, typically in kit or device form, useful for determining whether an individual or human cell is resistant to vinblastine or desoxyepothilone B. The kit may also be useful for inducing, conferring or otherwise imparting to a cell a resistance to an anti-microtubule agent such as vinblastine and desoxyepothilone B.

In one embodiment, the kit comprises, in an amount sufficient to perform at least one assay, at least one pair of primers comprising a first primer and a second primer capable of producing by PCR an amplification product that contains a mutation of the γ actin gene described above.

The primers are capable of amplifying a product from a provided nucleic acid sample. The primers are thus designed for the amplification of a preselected region of nucleic acid sequence to allow for the detection of any one of the mutations of the γ actin gene described above.

In another embodiment, the kit comprises, in an amount sufficient to perform at least one assay, at least one probe capable of detection of any one of the mutations of the γ actin gene described above.

In another embodiment, the kit comprises, in an amount sufficient to perform at least one assay, at least one antibody capable of detection of any one of the mutations of the γ actin gene described above.

The primers, probes or antibodies of the invention may be contained in separate containers in the kit.

The kit may also comprise instructions for use. “Instructions for use” typically include a tangible expression describing the reagent concentration or at least one assay method parameter such as the relative amounts of reagent and sample to be admixed, maintenance time periods for reagent/sample admixtures, use of control polynucleotide sequences, temperature, buffer conditions and the like.

The nucleic acid molecule, primers and probes of the kit may be labelled with a detectable label. Radioactive elements are useful labelling agents. An exemplary radiolabelling agent is a radioactive element that produces alpha ray emissions. Elements which themselves emit alpha rays, such as ³²p, ³⁵S, and ³³P represent one class of alpha ray emission-producing radioactive element indicating groups. Particularly preferred is ³²P. Also useful is a beta emitter, such ¹¹¹indium or ³²H. Non-radioactive methods include fluorescent, colourimetric, and chemiluminescent detection.

The antibody of the kit may also be labelled.

The components of the kit (i.e. including for example, nucleic acid molecules) can be provided in solution, as a liquid dispersion or as a substantially dry powder, e.g., in lyophilized form. The components of the kit may be contained in packaging materials such as those customarily utilised in diagnostic systems. For example, the packaging materials may be a solid matrix or material such as glass, plastic (e.g., polyethylene, polypropylene and polycarbonate), paper, foil and the like capable of holding within fixed limits a component or container for containing a component, of the kit. Thus, for example, a package can be a bottle, vial, plastic and plastic-foil laminated envelope or the like container used to contain a component of the kit.

The kit may comprise a carrier means being compartmentalised to receive in close confinement one or more containers such as vials, tubes, the like, each of the containers comprising one of more components.

### G. Producing Cells Resistant to an Anti-microtubule Agent.

As described herein, the inventor has found that the mutation of the γ actin gene and the γ actin protein expressed therefrom, confer resistance to anti-microtubule agents such as vinblastine, desoxyepothilone B and paclitaxel upon a cell. This finding has useful application particularly in the circumstance where there is a need to minimise or avoid the side effects that are observed in cancer therapy with these compounds.

Thus in one aspect, the invention provides a method for inducing, conferring or otherwise imparting to a cell, a resistance to an anti-microtubule agent. The method comprises the step of providing in a cell, a nucleic acid molecule encoding a mutant γ actin, or providing in a cell, a mutant γ actin.

In another aspect the invention provides a method for inducing, conferring or otherwise imparting to a cell, a resistance to vinblastine comprising providing in a cell:

(a) a γ actin wherein residue number 187 is histidine; or

(b) a γ actin wherein residue number 103 is leucine; or

(c) a nucleic acid molecule that encodes γ actin wherein nucleotide number 559 is cytosine; or

(d) a nucleic acid molecule that encodes γ actin wherein nucleotide number 307 is thymine.

In another aspect the invention provides a method for inducing, conferring or otherwise imparting to a cell, a resistance to desoxyepothilone B comprising providing in a cell:

(a) a γ actin wherein residue number 98 is leucine; or

(b) a γ actin wherein residue number 162 is methionine; or

(c) a nucleic acid molecule that encodes γ actin wherein nucleotide number 293 is thymine; or

(d) a nucleic acid molecule that encodes γ actin wherein nucleotide number 485 is thymine.

Typically the anti-microtubule agent is one selected from the group consisting of vinblastine, desoxyepothilone B, vincristine and paclitaxel.

Where a resistance to vinblastine is to be induced in a cell, typically the nucleic acid molecule comprises the sequence shown in any one of SEQ ID NO.s: 1 or 2. The mutant γ actin typically comprises the sequence shown in any one of SEQ ID NO.s: 5 or 6.

Where a resistance to desoxyepothilone B is to be induced in a cell, typically the mutated γ actin nucleic acid molecule comprises the sequence shown in any one of SEQ ID NO.s: 3 or 4. The mutated γ actin typically comprises the sequence shown in any one of SEQ ID NOS: 7 or 8.

Other examples of nucleic acid molecules that encode a mutant γ actin for use in this aspect of the invention are described above.

Typically, the mutated γ actin nucleic acid molecule is provided in the cell by introducing a vector or genetic construct comprising the nucleic acid molecule into the cell. The vector is preferably a plasmid or retrovirus vector. Vectors typically contain a promoter to direct transcription, a ribosome binding site for translational initiation, and a transcription/translation terminator. Examples of suitable vectors, processes for introducing a nucleic acid molecule to be expressed by the vector into the vector and processes for obtaining amounts of the vector useful for introducing the vector into a cell are described herein and in U.S. Pat. No. 5,851,819.

The cells in which resistance to an anti-microtubule agent is induced are mammalian cells, preferably human cells, more preferably bone marrow cells, embryonic stem cells, or cells of the gastrointestinal tract. Processes for introducing a nucleic acid molecule into bone marrow cells and other tissues and processes for identifying cells containing an introduced nucleic acid molecule are described herein and in U.S. Pat. No. 5,851,819.

Processes for identifying resistance to an anti-microtubule agent are described herein and also in U.S. Pat. No. 5,851,819.

In another aspect the invention provides a method for treating an individual for cancer with an anti-microtubule agent comprising providing in a non cancerous cell of the individual, a peptide having a sequence shown in any one of SEQ ID NO.s: 5 to 10, or a nucleic acid molecule comprising a sequence shown in any one of SEQ ID NOS: 1 to 4, to induce, confer or otherwise impart to the non cancerous cells, a resistance to anti-microtubule agent. In one embodiment the method comprises the step of isolating non cancerous cells from an individual, providing in the non cancerous cells a peptide having a sequence shown in any one of SEQ ID NOS: 5 to 10, or a nucleic acid molecule comprising a sequence shown in any one of SEQ ID NOS: 1 to 4, to induce, confer or otherwise impart to the non cancerous cells, a resistance to anti-microtubule agent, returning the isolated non cancerous cells to the individual and treating the individual with the anti-microtubule agent. The method may comprise the further step of monitoring the level of resistance of cells of the individual to an anti-microtubule agent.

### EXAMPLES

Experimental Procedures

Cell Culture

Human T-cell acute lymphoblastic leukaemia cells, CCRF-CEM, and drug resistant sublines, CEM/VCR R (vincristine-selected) and CEM/VLB100 (vinblastine-selected), and CEM/dEpoB 1×, CEM/dEpoB 2×, CEM/dEpoB 4×, CEM/dEpoB 10×(desoxyepothilone B-selected), were maintained in RPMI 1640 containing 10% FCS as suspension cultures. The CEM/VCRR are 22,600-fold and CEM/VLB100 are 6,667-fold resistant to vincristine and vinblastine respectively. Cells were harvested for protein analysis by centrifugation at 1500 rpm for 5 min.

Two-dimensional Polyacrylamide Gel Electrophoresis

Cells were washed three times in PBS and resuspended in buffer 1 (7M urea, 2M thiourea, 2% CHAPS, 1% sulfobetaine 3-10, 1% amidosulfobetaine-14, 2 mM TBP, 65 mM DTT, 1% carrier ampholyte 3-10, 1% carrier ampholyte 4-6, 0.01% bromophenol blue) to a final concentration of 1 mg/ml as determined by amino acid analysis. Cells were lysed by pulse sonication twice for 10 sec on ice. Endonuclease (Sigma) (1 unit/ug protein) was added and incubated at room temperature for 30 min. Protein extracts were centrifuged at 18,000×g for 12 min and the supernatant collected. Narrow range immobilised pH gradient (IPG) strips, pH 4.5-5.5 (Pharmacia, Upswala, Sweden), were rehydrated in 500 μl buffer 1. Protein (100 μg analytical, 500 μg preparative gels) was cup-loaded and isoelectric focused for 150,000 Vh on a Multiphor II apparatus (Pharmacia, Upswala, Sweden). IPG strips were equilibrated for 30 min in 6Murea, 2% SDS, 0.375M Tris-HCl (pH 8.8), 20% glycerol, 5 mM TBP, 2.5% acrylamide and embedded in 0.5% agarose on top of 20×18 cm 8-18%T polyacrylamide gels. SDSPAGE was performed at 20 mA/gel 16 hr at 4° C. Analytical gels were stained with SYPRO Ruby (Biorad, Calif.) or transferred to nitrocellulose (see below). Preparative gels were stained with colloidal coomassie G250. Relative levels of protein expression was determined on SYPRO Ruby stained gels using the Z3 Image Analysis program (Compugen Israel). All 2D-gel experiments were run in triplicate.

Immunoblotting

For specific protein detection analytical 2D-gels were transferred to nitrocellulose using standard methods. Actin was detected using polyclonal antibodies to total actin (1:2,000, Sigma), and a monoclonal antibody to 0-actin (1:5,000). Following incubation with a donkey anti-rabbit or sheep anti-mouse horseradish-peroxidase-linked antibody respectively, membranes were developed using Supersignal (Pierce, Rockford, Ill.).

MALDI-TOF Mass Spectrometry

Spots were excised from coomassie stained preparative gels, washed twice in 25 mM NH₄HCO₃, 50% AcN, spun dry and in gel trypsin digested in 10ηg/μl trypsin (Promega) in 25 mM NH₄HCO₃ for 16 hours at 37° C. Peptides were extracted from the gel with 50% (v/v) acetonitrile, 1% (v/v) TFA solution. A 1 μL aliquot was spotted onto a sample plate with 1 μL of matrix (α-cyano-4-hydroxcinnamic acid, 8 mg/mL in 50% v/v AcN, 1% v/v TFA). Matrix assisted laser desorption ionisation-time of flight (MALDI-TOF) mass spectrometry acquisition was performed on a TofSpec 2E mass spectrometer (Micromass, Manchester, UK) set to reflectron mode. Known trypsin auto-cleavage peptide masses (842.51Da; 2211.10Da) were used for a 2-point internal calibration for each spectrum.

ESI-TOF Tandem Mass Spectrometry

Upon analysis of MALDI-TOF mass spectra, peptides were selected for amino acid sequencing by ESI-TOF MS/MS. After in-gel trypsin digestion, the peptides were purified using a porous R2 resin column. The sample was then analysed by ESI-TOF MS/MS using a Micromass Q-TOF MS and data manually acquired using borosilicate capillaries for nanospray acquisition. Data was acquired over the m/z range 400-1800 Da to select peptides for MS/MS analysis. After peptides were selected, the MS was switched to MS/MS mode and data collected over the m/z range 50-2000 Da with variable collision energy settings.

Partial Sequencing of γ Actin cDNA

Partial sequencing of γ-actin cDNA was performed in CCRF/CEM, CEM/VCR R, CEM/VLB100, and the CEM/dEpoB 4×cells using primers G-Act 1F: ATGGAAGAAGAGATCGCCGC and G-Act 654R: TCGGCCGTGGTGGTGAA. cDNA was amplified in 1×amplification buffer, 0.75 mM dNTPs, 62 ηg G-Act 1F, 62 ηg G-Act 654R, pfu-turbo polymerase in a total reaction volume of 20 μl, using the following PCR cycling: 96° C. 10 min, 35×(96° C, 1 min, 64° C. 1 min, 72° C. 45 sec), 72° C. 10 min. Amplified cDNA was purified using the QIA Filter Gel Extraction Kit (Quiagen, Hilden, Germany) and sequenced using fluorescence based cycle sequencing with BigDye terminators (PE Biosystems, Foster City, Calif.). Sequences were analysed by the Automated Sequencing Facility, Biological Sciences, University of NSW. The γ actin sequence of the resistant cell lines was compared with that of the parental CEM cell line and the published sequence.

Modeling of γ-actin Structure

Actin modelling was generated from the actin structure (PDB# 1ATN) using the Insight II modelling program (Molecular Simulations Inc.).

Fluorescent Microscopy

Cells were centrifuged onto glass slides and fixed in 3.7% formaldehyde for 10 min and permeabilized in 0.1% triton X-100 for 3 min. For detection of total filamentous actin, cells were incubated in 5 μg/ml FITC-Phalloidin for 10 min. For immunofluorescence, cells were blocked in 10% FCS/PBS for 20 min, and incubated in primary antibody (1:250 γ-actin; 1:500 α-tubulin) at 37° C. in a humidified chamber for 45 min. After repeated washes cells were incubated in secondary antibody (1:1000 anti-Sheep Alexa 488 conjugate and/or Rabbit-anti-mouse Cy3 conjugate). All slides were washed extensively in PBS or 5% FCS/PBS between each step. Finally slides were mounted with DAPI for nuclear staining. Epifluorescence imaging was performed using a Zeiss Axioscope 100 and a cooled CCD camera.

Separation of Soluble and Filamentous Actin.

G-actin plus short actin filaments (soluble) and the larger actin filaments (insoluble) were separated by detergent extraction and centrifugation, with or without the addition of 5 μM cytochalasin D to the lysis buffer. Relative levels of soluble and insoluble actin were detected by immunoblotting with a total actin antibody. Percentage insoluble actin was calculated as the densitometric value of the insoluble fraction divided by the total densitometric value of the insoluble plus soluble fraction of three independent experiments. Construction of Mutant γ-actin Expression Plasmids.

Site directed mutagenesis for each of the four γ-actin mutations was performed on a full length γ-actin cDNA sequence in the Okayama-Berg vector, using the GeneEditor™ in vitro site-directed mutagenesis system (Promega) according to the manufacturers instructions. The γ-actin sequence was then cloned into the pcDNA3.1(−) vector using standard procedures.

Stable Transfection of γ-actin Constructs.

DNA (1 μg) was transfected into subconfluent NIH3T3 mouse fibroblast cells using the Fugene (Roche) transfection reagent according to the manufacturer's instructions. Stable transfectants were selected in 1 mgm1⁻¹ G418 for 16 days. Levels of γ-actin expression were determined by immunoblotting as described above.

Drug Resistance Assays.

The ability of mutant γ-actin to confer resistance to antimicrotubule agents was determined using clonogenic assays with slight modifications. NIH3T3 mutant γ-actin transfectant cells were seeded at 100 cells/well in replicate wells of a 24-well dish containing increasing concentrations of VLB. The media was replaced after 3 days with drug-free DME/10%FCS. After a further 4 days the media was removed and colonies stained with 0.1% crystal violet. The densitometric volume of each well was determined using the Quantity One software package (Biorad) and expressed relative to the respective drug-free control cells. The concentration that kills 50% of cells (ID50) and statistical analyses were then performed. The relative resistance was determined by dividing the ID50 of each γ-actin transfectant cell line with the ID50 of the empty vector cells.

Drug Treatment and Immunofluorescence.

NIH3T3 mutant γ-actin transfectant cells were grown to ˜80% confluency in 4-well chamber slides (Nalge Nunc International, Naperville, Ill.) and treated with either 0.5% DMSO (vehicle control), 10 μM VLB, or 10 μM dEpoB, for 30 min, prior to immunostaining for total α-tubulin using standard methods. Epifluorescence imaging was performed using a Zeiss Axioscope 100 linked to a cooled CCD camera.

Results & Discussion

To investigate the mechanisms involved in resistance to vinblastine or desoxyepothilone B (dEpoB), the differential gene and protein expression between vinblastine or dEpoB —sensitive and —resistant cell lines was studied.

1. Numerous Protein Changes are Associated with Vinblastine Resistant ALL Cell Lines.

Total cellular proteins from CEM, VCRR and VLB100 cell lines were separated by 2DPAGE. The use of overlapping narrow pH ranges allowed for the separation of 8575 proteins, in contrast to 1136 proteins separated in the same pH range on a broad 3-10 IPG (Table 1). Analysis of proteins in the pH 4.5-5.5 region identified numerous differentially expressed proteins between the drug sensitive and drug resistant cell lines.

2. Vinblastine Resistant Leukaemia Cells Express an Extra More Basic Actin Isoform.

Of particular interest was the high level expression of a γ-actin isoform observed only in the VLB100 cells spot 19 (data not shown). This protein was initially identified as γ-actin by PMF. β- and γ-actin differ in only four residues at their N-terminus, thus confirmation of the PMF identification was obtained by MS/MS sequencing of the N terminal peptide (peptide 1912.8Da) in spot 19 (data not shown). The sequence matched to that of γ-actin. Spot 15 was identified as β-actin and spot 16 as γ-actin in all three cell lines by PMF (data not shown). Further confirmation was obtained by immunoblotting. β-actin was detected as a single protein spot in all three cell lines (data not shown). However, when a pAb for total actin is used, the spot corresponding to γ-actin protein is detected in CEM and VCR R cells, whilst two distinct γ-actin spots are detected in VLB100 cells (data not shown).

The expression levels of the three major actin spots were measured (data not shown). β- and γ-actin in CEM and VCR R cells are expressed at very similar levels. The same isoelectric forms of β- and γ-actin in VLB100 cells show a 1.7 and 2.0 fold decrease in expression respectively, compared to CCRF-CEM. Although VLB100 cells express an extra isoform, γ′-actin, the total actin expression in not significantly changed compared to the CEM or VCR R cells.

β- and γ-actin can both regulate the expression of their co-expressed isoforms, thus the decrease in β- and γ-actin isoforms in the VLB100 cells is likely to be in compensation for the high expression of the γ′-actin isoform. However, the ratio of β- to γ-actin is highly regulated and tissue specific in normal cells, suggesting that the two isoforms have functional diversity. Alterations in this ratio have been identified in the human ALL cell line, MOLT4, with an increase in expression of γ-actin to β-actin than normal T-lymphocytes and in chemically transformed human osteosarcoma HOS fibroblasts with a concomitant increase in tumorigenic potential. The ratio of β- to γ-actin in the CEM and VLB100 cells is 2.7:1. Including both γ- and γ′-actin isoforms, the ratio of β- to γ-actin in VLB100 cells is 1:1.

3. Vinblastine Resistant Cells Express two Mutant γ-actin Proteins.

The approximate pI for the β-actin spot is 5.29, for the “normal” β-actin is 5.31, and for the spot 19 found only in the VLB100 cells is 5.46. To determine the cause of the isoelectric shift seen for spot 19, MS/MS was performed on peptides showing different masses between the γ- and γ′-actin. Two peptide mass differences were seen by MALDI-TOF MS: peptides at 1968.1Da and 998.5Da in γ-actin; peptides at 1020.6 and 1954.1Da in γ′-actin (data not shown). The amino acid sequence of these peptides shows that peptide 998.5Da, DLTDYLMK, from spot 16VLB100 γ-actin matches to the published γ- actin sequence aa184 to 191, whilst the 1020.6Da peptide from VLB100 γ′-actin (spot19), DLTHYLMK, was the same peptide with a mutation of D₁₈₇→H₁₈₇. Similarly, peptide 1954.1Da from γ′-actin (spot 19), VAPEEHPVLLTEAPLNPK, matched the published sequence between residues 96 to 113, and 1968.1Da from spot 16 VAPEEHPL/ILLTEAPLNPK was found to be the same peptide but with a mutation of V₁₀₃→L/I₁₀₃. As leucine and isoleucine differ by 0.01Da mass, these two amino acids cannot be distinguished using MS/MS. Sequencing of the 998.5Da and 1954.1Da peptides in CEM and VCR R revealed no changes from the published sequence. Confirmation of these mutations was obtained by cDNA sequencing (data not shown). cDNA sequencing of γ-actin from these cell lines detected heterozygous mutations G→C (D₁₈₇→H₁₈₇), and G→T (V₁₀₃→L₁₀₃). A silent mutation of G→A was also identified in VLB100 cells. No differences were found in the CEM and VCRR cells to that of the published sequence. Thus γ′-actin (spot 19) harbours a substitution of aspartic acid, a positively charged amino acid, for histidine, a neutral amino acid, resulting in a more basic isoelectric point and hence the basic charge shift seen by 2D-PAGE. The substitution of a valine to a leucine does not change the charge on the protein and thus VLB100 γ-actin (spot 16) migrates to the same position as the wild type γ-actin in CEM and VCRR cells.

There have been no reports to date of γ-actin mutations associated with drug resistance. A mutant γ-actin was found in the human promyelocytic cell line, HL60. Expression of a mutant β-actin (Gly₂₄₄→Asp244) was identified in a transformed, tumorigenic fibroblast cell line, Hut-14. Transfection of this mutant β-actin into a transformed, but non-tumirogenic cell line, induced morphological changes and tumourigenicity in these cells, with a positive correlation of mutant β-actin expression and extent of tumorigenicity. A variant β-actin has also been linked to decreased metastasis of mouse B16 melanoma. Numerous alterations in actin-binding proteins have also been associated with cell transformation and tumorigenesis.

4. The D₁₈₇″H₁₈₇ Mutation in VLB100 γ-actin Resides in the ATP-binding Domain.

Modelling of the γ-actin mutations revealed that D₁₈₇→H187 resides within subdomain IV (data not shown). As subdomain IV is at small radius within the filament, it has been suggested that even minor changes in this domain may have major structural and stability effects on the polymer. This mutation is also in close proximity to the ATP binding cleft of the protein. The V₁₀₃→L₁₀₃ mutation lies within subdomain I. This is thought to be the site of various actin-binding proteins.

5. The Actin Cytoskeleton is Structurally Different in Vinblastine Resistant Cells.

As multiple changes in actin expression were identified in the vinblastine resistant cells, we analysed the structure of the actin cytoskeleton by fluorescent microscopy (data not shown). Staining for total filamentous actin revealed marked differences between the three cell lines. All cells exhibit filamentous extensions, or microspikes, protruding from the plasma membrane, however, in the VLB100 cells, they are now much thinner, longer, and more hair-like than the parental cells. Specifically staining for γ-actin shows similar cytoskeletal structures as total actin staining in the CEM cells. The VLB100 cells display very few visible microspikes with the γ-actin antibody, and have a granulated staining appearance. As a control for cytoskeletal integrity, cells were also stained for α-tubulin (data not shown).

6. Detection of γ-actin Mutations in Desoxyepothilone B Resistant CEM Cells.

Desoxyepothilone B (dEpoB) is a relatively new anti-microtubule agent showing great promise in vivo. dEpoB shares a common cellular mechanism of action with taxol, stabilizing microtubules thus leading to mitotic arrest and eventually cell death. One of the most important properties of the epothilones is their lack of cross-resistance to multi-drug resistant cells. Thus cell lines resistant to dEpoB must have alterations other than Pgp, giving rise to the resistance phenotype. To understand the mechanisms of resistance to dEpoB, we developed a range of resistant cell lines to this agent. 2D-PAGE analysis of CEM cells selected for resistance to 130 μM dEpoB detected a new γ-actin spot at slightly lower molecular weight than the wild type γ-actin in the parental CEM cells. This isoform is expressed at similar levels to the higher molecular weight isoform. cDNA sequencing identified two heterozygous mutations in these cells (P₉₈→L₉₈; T₁₆₂→M₁₆₂. These mutations were confirmed at the protein level by mass spectrometry. The wild type sequence has a peptide mass of 1954.1 Da and the mutant P98L is 1970.1. The T162M mutant has a peptide at 3245.6 in contrast to the wildtype at 3199.6. The p98L mutation is just 5 residues away from the V103L mutation found in the VLB100 cells. The T162M is 25 residues from D187H in VLB100 cells, however still resides within subdomain IV and is in closer proximity to the ATP binding site. Thus we have identified two mutations in γ-actin in two ALL cell lines selected for resistance to anti-microtubule agents. This is the first report of γ-actin mutations associated with drug resistance.

7. Inducing Resistance to Anti-microtubule Agents with Mutant γ Actin.

To test the functional significance of these mutations we transfected each γ-actin mutant, together with the two mutant combinations identified in the resistant cells, into NIH3T3 mouse fibroblast cells. Expression of either wild-type or mutant γ-actin resulted in only a modest increase in the expression of total γ-actin (data not shown). This is to be expected because the levels of actin in the cell are subject to autoregulation and expression from the exogenous gene is competing with the endogenous β- and γ-actin genes for contribution to the total actin pool. Importantly, expression of mutant γ-actin was sufficient to provide resistance to anti microtubule drug treatment (data not shown). Cells transfected with mutant γ-actin display far greater clonogenic cell survival than either the empty vector or wildtype γ-actin cells (data not shown). The P98L mutant is over 3-fold (P<0.05) and V103L mutant is over 2-fold resistance (P<0.05) to VLB compared to the empty vector and wildtype γ-actin cells (data not shown). Cells expressing the double mutants show similar resistance as the P98L and V103L mutants alone, suggesting that the mutations do not have an additive effect and that the mutations in subdomain 1 of γ-actin (data not shown) play a predominant role in resistance to antimicrotubule drugs. The T162M and D187H mutants exhibit similar survival as the control cells (data not shown). This may be because a higher expression of these mutants is required for an observable effect. Similar resistance profiles were observed in cells treated with dEpoB or with paclitaxel (data not shown) indicating that the γ-actin mutations provide resistance to a wide variety of anti microtubule agents.

To investigate how the γ-actin mutations protect against the action of anti-microtubule agents, cells were treated with vinblastine or dEpoB and the microtubules analysed by fluorescence microscopy (data not shown). A normal microtubule network is observed in all untreated cells. In empty vector and wild type expressing cells, both VLB and dEpoB causes marked structural changes. The cell shape is disrupted and extensive cell rounding is observed. Remarkably the mutant γ-actin transfectants retain their basic cell shape and substratum adhesion, and the microtubule network remains largely intact following exposure to these anti microtubule drugs (data not shown). Interestingly, the T162M and D187H mutants also retain their cell shape however these cells did not show significant resistance using the clonogenic assay. These results indicate that wild type γ-actin is required for anti microtubule-induced cell contraction and cell death, or that the mutations in γ-actin are able to provide structural support to the microtubules, which in turn resist the impact of the antimicrotubule drugs. This is the first evidence that mutations in actin directly affect the response of cells to antimicrotubule agents and demonstrate that alterations in the actin cytoskeleton can profoundly affect the dynamics of the microtubule system.

Table 1. Total number of spots separated in each pH range 2D-gel

TABLE 1
Total number of spots separated in each pH range 2D-gel

| PH Range 2D-gel | No. spots |
|---|---|
| 3-10 | 1136 |
| 4-7 | 2428 |
| 4.5-5.5 | 2238 |
| 5.0-6.0 | 3047 |
| 5.5-6.7 | 1826 |
| 6.0-9.0 | 1464 |

1-8. (canceled)

9. A method for inducing in a cell a resistance to an anti-microtubule agent comprising the step of providing in a cell, a nucleic acid molecule that encodes a mutant γ actin, or providing in a cell, a mutant γ actin.

10. A method for producing a cell that is resistant to an anti-microtubule agent comprising the step of providing in a cell, a nucleic acid molecule that encodes a mutant γ actin, or providing in a cell, a mutant γ actin.

11. A method for treating an individual for cancer with an anti-microtubule agent comprising the step of providing in a non cancerous cell of the individual, a nucleic acid molecule that encodes a mutant γ actin, or providing in a non cancerous cell, a mutant γ actin, to induce in the non cancerous cell, a resistance to an anti-microtubule agent.

12. A method for determining whether a compound is capable of treating a cell having a drug resistance phenotype comprising the steps of:
providing in a cell a nucleic acid molecule that encodes a mutant γ actin, or providing in a cell, a mutant γ actin, to induce in the cell, a resistance to an anti-microtubule agent; and
contacting the cell with the compound to determine whether the compound is capable of treating the cell.

13. A peptide for inducing in a cell a resistance to an anti-microtubule agent comprising:
(a) a sequence shown in any one of SEQ ID NO.s: 5 to 10; or
(b) a sequence that has at least 80% identity to the sequence shown in SEQ ID NO: 5 and wherein residue number 187 is histidine; or
(c) a sequence that has at least 80% identity to the sequence shown in SEQ ID NO: 6 and wherein residue number 103 is leucine; or
(d) a sequence that has at least 80% identity to the sequence shown in SEQ ID NO: 7 and wherein residue number 98 is leucine; or
(e) a sequence that has at least 80% identity to the sequence shown in SEQ ID NO: 8 and wherein residue number 162 is methionine; or
(f) a fragment of a peptide according to any one of (a) to (e).

14. A nucleic acid molecule that encodes a peptide according to claim 13.

15. A nucleic acid molecule for inducing in a cell a resistance to an anti-microtubule agent comprising:
(a) a sequence shown in any one of SEQ ID NO.s: 1 to 4; or
(b) a sequence that has at least 80% identity to the sequence shown in SEQ ID NO: 1 and wherein nucleotide number 559 is cytosine; or
(c) a sequence that has at least 80% identity to the sequence shown in SEQ ID NO: 2 and wherein nucleotide number 307 is thymine; or
(d) a sequence that has at least 80% identity to the sequence shown in SEQ ID NO: 3 and wherein nucleotide number 293 is thymine; or
(e) a sequence that has at least 80% identity to the sequence shown in SEQ ID NO: 4 and wherein nucleotide number 485 is thymine; or
(f) a fragment of a molecule according to any one of (a) to (e).


SEQUENCE LISTING (10 sequences)

SEQ ID NO: 1 - DNA, 1845 bp, Homo sapiens
atggaagaagagatcgccgcgctggtcattgacaatggctccggcatgtgcaaagctggttttgctggggacgacgctccccgagccgtgtttccttccatcgtcgggcgccccagacaccagggcgtcatggtgggcatgggccagaaggactcctacgtgggcgacgaggcccagagcaagcgtggcatcctgaccctgaagtaccccattgagcatggcatcgtcaccaactgggacgacatggagaagatctggcaccacaccttctacaacgagctgcgcgtggccccggaggagcacccagtgctgctgaccgaggcccccctgaaccccaaggccaacagagagaagatgactcagattatgtttgagaccttcaacaccccggccatgtacgtggccatccaggccgtgctgtccctctacgcctctgggcgcaccactggcattgtcatggactctggagacggggtcacccacacggtgcccatctacgagggctacgccctcccccacgccatcctgcgtctggacctggctggccgggacctgacccactacctcatgaagatcctcactgagcgaggctacagcttcaccaccacggccgagcgggaaatcgtgcgcgacatcaaggagaagctgtgctacgtcgccctggacttcgagcaggagatggccaccgccgcatcctcctcttctctggagaagagctacgagctgcccgatggccaggtcatcaccattggcaatgagcggttccggtgtccggaggcgctgttccagccttccttcctgggtatggaatcttgcggcatccacgagaccaccttcaactccatcatgaagtgtgacgtggacatccgcaaagacctgtacgccaacacggtgctgtcgggcggcaccaccatgtacccgggcattgccgacaggatgcagaaggagatcaccgccctggcgcccagcaccatgaagatcaagatcatcgcacccccagagcgcaagtactcggtgtggatcggtggctccatcctggcctcactgtccaccttccagcagatgtggattagcaagcaggagtacgacgagtcgggcccctccatcgtccaccgcaaatgcttctaaacggactcagcagatgcgtagcatttgctgcatgggttaattgagaatagaaatttgcccctggcaaatgcacacacctcatgctagcctcacgaaactggaataagccttcgaaaagaaattgtccttgaagcttgtatctgatatcagcactggattgtagaacttgttgctgattttgaccttgtattgaagttaactgttccccttggtatttgtttaataccctgtacatatctttgagttcaacctttagtacgtgtggcttggtcacttcgtggctaaggtaagaacgtgcttgtggaagacaagtctgtggcttggtgagtctgtgtggccagcagcctctgatctgtgcagggtattaacgtgtcagggctgagtgttctgggatttctctagaggctggcaagaaccagttgttttgtcttgcgggtctgtcagggttggaaagtccaagccgtaggacccagtttcctttcttagctgatgtctttggccagaacaccgtgggctgttacttgctttgagttggaagcggtttgcatttacgcctgtaaatgtattcattcttaatttatgtaaggttttttttgtacgcaattctcgattctttgaagagatgacaacaaattttggttttctactgttatgtgagaacattaggccccagcaacacgtcattgtgtaaggaaaaataaaagtgctgccgtaacc

SEQ ID NO: 2 - DNA, 1845 bp, Homo sapiens
atggaagaagagatcgccgcgctggtcattgacaatggctccggcatgtgcaaagctggttttgctggggacgacgctccccgagccgtgtttccttccatcgtcgggcgccccagacaccagggcgtcatggtgggcatgggccagaaggactcctacgtgggcgacgaggcccagagcaagcgtggcatcctgaccctgaagtaccccattgagcatggcatcgtcaccaactgggacgacatggagaagatctggcaccacaccttctacaacgagctgcgcgtggccccggaggagcacccattgctgctgaccgaggcccccctgaaccccaaggccaacagagagaagatgactcagattatgtttgagaccttcaacaccccggccatgtacgtggccatccaggccgtgctgtccctctacgcctctgggcgcaccactggcattgtcatggactctggagacggggtcacccacacggtgcccatctacgagggctacgccctcccccacgccatcctgcgtctggacctggctggccgggacctgaccgactacctcatgaagatcctcactgagcgaggctacagcttcaccaccacggccgagcgggaaatcgtgcgcgacatcaaggagaagctgtgctacgtcgccctggacttcgagcaggagatggccaccgccgcatcctcctcttctctggagaagagctacgagctgcccgatggccaggtcatcaccattggcaatgagcggttccggtgtccggaggcgctgttccagccttccttcctgggtatggaatcttgcggcatccacgagaccaccttcaactccatcatgaagtgtgacgtggacatccgcaaagacctgtacgccaacacggtgctgtcgggcggcaccaccatgtacccgggcattgccgacaggatgcagaaggagatcaccgccctggcgcccagcaccatgaagatcaagatcatcgcacccccagagcgcaagtactcggtgtggatcggtggctccatcctggcctcactgtccaccttccagcagatgtggattagcaagcaggagtacgacgagtcgggcccctccatcgtccaccgcaaatgcttctaaacggactcagcagatgcgtagcatttgctgcatgggttaattgagaatagaaatttgcccctggcaaatgcacacacctcatgctagcctcacgaaactggaataagccttcgaaaagaaattgtccttgaagcttgtatctgatatcagcactggattgtagaacttgttgctgattttgaccttgtattgaagttaactgttccccttggtatttgtttaataccctgtacatatctttgagttcaacctttagtacgtgtggcttggtcacttcgtggctaaggtaagaacgtgcttgtggaagacaagtctgtggcttggtgagtctgtgtggccagcagcctctgatctgtgcagggtattaacgtgtcagggctgagtgttctgggatttctctagaggctggcaagaaccagttgttttgtcttgcgggtctgtcagggttggaaagtccaagccgtaggacccagtttcctttcttagctgatgtctttggccagaacaccgtgggctgttacttgctttgagttggaagcggtttgcatttacgcctgtaaatgtattcattcttaatttatgtaaggttttttttgtacgcaattctcgattctttgaagagatgacaacaaattttggttttctactgttatgtgagaacattaggccccagcaacacgtcattgtgtaaggaaaaataaaagtgctgccgtaacc

SEQ ID NO: 3 - DNA, 1845 bp, Homo sapiens
atggaagaagagatcgccgcgctggtcattgacaatggctccggcatgtgcaaagctggttttgctggggacgacgctccccgagccgtgtttccttccatcgtcgggcgccccagacaccagggcgtcatggtgggcatgggccagaaggactcctacgtgggcgacgaggcccagagcaagcgtggcatcctgaccctgaagtaccccattgagcatggcatcgtcaccaactgggacgacatggagaagatctggcaccacaccttctacaacgagctgcgcgtggccctggaggagcacccagtgctgctgaccgaggcccccctgaaccccaaggccaacagagagaagatgactcagattatgtttgagaccttcaacaccccggccatgtacgtggccatccaggccgtgctgtccctctacgcctctgggcgcaccactggcattgtcatggactctggagacggggtcacccacacggtgcccatctacgagggctacgccctcccccacgccatcctgcgtctggacctggctggccgggacctgaccgactacctcatgaagatcctcactgagcgaggctacagcttcaccaccacggccgagcgggaaatcgtgcgcgacatcaaggagaagctgtgctacgtcgccctggacttcgagcaggagatggccaccgccgcatcctcctcttctctggagaagagctacgagctgcccgatggccaggtcatcaccattggcaatgagcggttccggtgtccggaggcgctgttccagccttccttcctgggtatggaatcttgcggcatccacgagaccaccttcaactccatcatgaagtgtgacgtggacatccgcaaagacctgtacgccaacacggtgctgtcgggcggcaccaccatgtacccgggcattgccgacaggatgcagaaggagatcaccgccctggcgcccagcaccatgaagatcaagatcatcgcacccccagagcgcaagtactcggtgtggatcggtggctccatcctggcctcactgtccaccttccagcagatgtggattagcaagcaggagtacgacgagtcgggcccctccatcgtccaccgcaaatgcttctaaacggactcagcagatgcgtagcatttgctgcatgggttaattgagaatagaaatttgcccctggcaaatgcacacacctcatgctagcctcacgaaactggaataagccttcgaaaagaaattgtccttgaagcttgtatctgatatcagcactggattgtagaacttgttgctgattttgaccttgtattgaagttaactgttccccttggtatttgtttaataccctgtacatatctttgagttcaacctttagtacgtgtggcttggtcacttcgtggctaaggtaagaacgtgcttgtggaagacaagtctgtggcttggtgagtctgtgtggccagcagcctctgatctgtgcagggtattaacgtgtcagggctgagtgttctgggatttctctagaggctggcaagaaccagttgttttgtcttgcgggtctgtcagggttggaaagtccaagccgtaggacccagtttcctttcttagctgatgtctttggccagaacaccgtgggctgttacttgctttgagttggaagcggtttgcatttacgcctgtaaatgtattcattcttaatttatgtaaggttttttttgtacgcaattctcgattctttgaagagatgacaacaaattttggttttctactgttatgtgagaacattaggccccagcaacacgtcattgtgtaaggaaaaataaaagtgctgccgtaacc

SEQ ID NO: 4 - DNA, 1845 bp, Homo sapiens
atggaagaagagatcgccgcgctggtcattgacaatggctccggcatgtgcaaagctggttttgctggggacgacgctccccgagccgtgtttccttccatcgtcgggcgccccagacaccagggcgtcatggtgggcatgggccagaaggactcctacgtgggcgacgaggcccagagcaagcgtggcatcctgaccctgaagtaccccattgagcatggcatcgtcaccaactgggacgacatggagaagatctggcaccacaccttctacaacgagctgcgcgtggccccggaggagcacccagtgctgctgaccgaggcccccctgaaccccaaggccaacagagagaagatgactcagattatgtttgagaccttcaacaccccggccatgtacgtggccatccaggccgtgctgtccctctacgcctctgggcgcaccactggcattgtcatggactctggagacggggtcacccacatggtgcccatctacgagggctacgccctcccccacgccatcctgcgtctggacctggctggccgggacctgaccgactacctcatgaagatcctcactgagcgaggctacagcttcaccaccacggccgagcgggaaatcgtgcgcgacatcaaggagaagctgtgctacgtcgccctggacttcgagcaggagatggccaccgccgcatcctcctcttctctggagaagagctacgagctgcccgatggccaggtcatcaccattggcaatgagcggttccggtgtccggaggcgctgttccagccttccttcctgggtatggaatcttgcggcatccacgagaccaccttcaactccatcatgaagtgtgacgtggacatccgcaaagacctgtacgccaacacggtgctgtcgggcggcaccaccatgtacccgggcattgccgacaggatgcagaaggagatcaccgccctggcgcccagcaccatgaagatcaagatcatcgcacccccagagcgcaagtactcggtgtggatcggtggctccatcctggcctcactgtccaccttccagcagatgtggattagcaagcaggagtacgacgagtcgggcccctccatcgtccaccgcaaatgcttctaaacggactcagcagatgcgtagcatttgctgcatgggttaattgagaatagaaatttgcccctggcaaatgcacacacctcatgctagcctcacgaaactggaataagccttcgaaaagaaattgtccttgaagcttgtatctgatatcagcactggattgtagaacttgttgctgattttgaccttgtattgaagttaactgttccccttggtatttgtttaataccctgtacatatctttgagttcaacctttagtacgtgtggcttggtcacttcgtggctaaggtaagaacgtgcttgtggaagacaagtctgtggcttggtgagtctgtgtggccagcagcctctgatctgtgcagggtattaacgtgtcagggctgagtgttctgggatttctctagaggctggcaagaaccagttgttttgtcttgcgggtctgtcagggttggaaagtccaagccgtaggacccagtttcctttcttagctgatgtctttggccagaacaccgtgggctgttacttgctttgagttggaagcggtttgcatttacgcctgtaaatgtattcattcttaatttatgtaaggttttttttgtacgcaattctcgattctttgaagagatgacaacaaattttggttttctactgttatgtgagaacattaggccccagcaacacgtcattgtgtaaggaaaaataaaagtgctgccgtaacc

SEQ ID NO: 5 - protein, 375 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTHYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 6 - protein, 375 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPLLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 7 - protein, 375 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVALEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 8 - protein, 375 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHMVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 9 - protein, 8 aa, Homo sapiens
DLTHYLMK

SEQ ID NO: 10 - protein, 18 aa, Homo sapiens
VAPEEHPVLLTEAPLNPL